(19) United States
(12) Patent Application Publication
(10) Pub. No.: US 2023/0072788 A1
(43) Pub. Date: Mar. 9, 2023

(54) Uses of 5-Aminovaleric Acid Betaine and Compositions Related Thereto

(71) Applicant: Emory University, Atlanta, GA (US)

(72) Inventors: Dean P. Jones, Decatur, GA (US); Andrew S. Neish, Atlanta, GA (US); Ken Liu, Atlanta, GA (US); Bejan Saeedi, Atlanta, GA (US)

(21) Appl. No.: 17/903,392

(22) Filed: Sep. 6, 2022

Related U.S. Application Data
(60) Provisional application No. 63/240,555, filed on Sep. 3, 2021.

(51) Int. Cl. G01N 33/92; A23K 20/158; G01N 33/68

(52) U.S. Cl. CPC G01N 33/92; A23K 20/158; G01N 33/68; G01N 2800/32; G01N 2800/50; G01N 2800/044

(57) ABSTRACT

This disclosure relates to uses of 5-aminovaleric acid betaine and compositions related thereto. In certain embodiments, this disclosure relates to diagnostic assays and methods of measuring and monitoring 5-aminovaleric acid betaine levels or the ratio 5-aminovaleric acid betaine to carnitine in a sample. In certain embodiments, this disclosure relates to methods of treating or preventing muscle wasting comprising administering to a subject in need thereof an effective amount of 5-aminovaleric acid betaine, prodrug, or salt thereof

### CROSS-REFERENCE TO RELATED APPLICATIONS

This application claims the benefit of U.S. Provisional Application No. 63/240,555 filed Sep. 3, 2021. The entirety of this application is hereby incorporated by reference for all purposes.

### STATEMENT REGARDING FEDERALLY SPONSORED RESEARCH OR DEVELOPMENT

This invention was made with government support under ES023485, ES025632, DK117570, and AI064462 awarded by the National Institutes of Health. The government has certain rights in the invention.

### BACKGROUND

A world-wide epidemic of obesity is ongoing with important demographic shifts implying a near universal health challenge. With complex contributing factors, emerging evidence links the epidemic to unfavorable changes in the composition and activity of the intestinal microbiota. Given the complexity of microbiome, diet and genetics, new technologies are needed to reduce obesity. Metabolomic profiling complements metagenomic methods with the capability to measure metabolic products of microbiome, diet, and host interactions.

Hoppel et al. describe proposed pathways for lysine metabolism that results in betaines such as carnitine and 5-aminovaleric acid betaine. Biochem. J. (1980) 188, 509-519.

Kärkkäinen et al. report 5-aminovaleric acid betaine decreases (3-oxidation of fatty acids in mouse cardiomyocytes. Sci Rep 8, 13036 (2018). See also WO2018/150077.

Zhao et al. report N,N,N-trimethyl-5-aminovaleric acid is increased in plasma from patients with liver steatosis, inhibits y-butyrobetaine hydroxylase, and exacerbates fatty liver in mice. Gastroenterology, 2020, 158(8):2266-2281.

References cited herein are not an admission of prior art.

### SUMMARY

This disclosure relates to uses of 5-aminovaleric acid betaine and compositions related thereto. In certain embodiments, this disclosure relates to diagnostic assays and methods of measuring and monitoring 5-aminovaleric acid betaine levels or the ratio 5-aminovaleric acid betaine to carnitine in a sample. In certain embodiments, this disclosure relates to methods of treating or preventing muscle wasting comprising administering to a subject in need thereof an effective amount of 5-aminovaleric acid betaine, prodrug, or salt thereof.

In certain embodiments, this disclosure relates to methods of treating or preventing obesity comprising measuring the amount of 5-aminovaleric acid betaine in a sample from a subject optionally in combination with measuring the amount of carnitine in the sample and treating or advising the subject to take certain actions to treat or prevent obesity. In certain embodiments, treating is administering or prescribing a weight loss therapeutic or dietary formulation optionally in combination with advising a schedule of feeding, exercise, and/or a sleep schedule.

In certain embodiments, this disclosure relates to methods of diagnosing a subject for a risk level of obesity complications comprising, obtaining a sample from a subject; measuring an amount of 5-aminovaleric acid betaine in the sample; and diagnosing the subject with a risk level for of obesity complications based on the measured amount 5-aminovaleric acid betaine in the sample.

In certain embodiments, this disclosure relates to methods of diagnosing a subject for a risk level of obesity complications comprising, obtaining a sample from a subject; measuring an amount of 5-aminovaleric acid betaine in the sample; measuring an amount of carnitine in the sample; calculating the ratio of 5-aminovaleric acid betaine to carnitine in the sample; and diagnosing the subject with a risk level for obesity complications based on the calculated ratio of 5-aminovaleric acid betaine to carnitine in the sample.

In certain embodiments, this disclosure relates to methods of diagnosing a subject with 5-aminovaleric acid betaine producing obesogenic microbiota or for a risk level of 5-aminovaleric acid betaine related obesity complications comprising: obtaining a sample from a subject; measuring an amount of 5-aminovaleric acid betaine in the sample; measuring an amount of carnitine in the sample; calculating the ratio of 5-aminovaleric acid betaine to carnitine in the sample; and diagnosing the subject with a risk level for of 5-aminovaleric acid betaine related obesity complications based on the calculated ratio of 5-aminovaleric acid betaine to carnitine in the sample.

In certain embodiments, this disclosure relates to methods of evaluating the effects of a test compound for the ability to treat or prevent obesity complications comprising administering a test compound to a subject; obtaining a sample from the subject; measuring an amount of 5-aminovaleric acid betaine in the sample; measuring an amount of carnitine in the sample; calculating the ratio of 5-aminovaleric acid betaine to carnitine in the sample; and evaluating whether the test compound provides a reduced risk level for obesity complications based on a reduction of the amount of 5-aminovaleric acid betaine when compared to a normal or reference value, e.g., when compared to a subject that did not receive an administration of the test compound, or a reduction in the calculated ratio of 5-aminovaleric acid betaine to carnitine in the sample when compared to a subject that did not receive an administration of the test compound.

In certain embodiments, this disclosure contemplates dietary formulations with custom-blended dietary supplements for pre/probiotic modulation of 5-aminovaleric acid betaine production and supplement formulations containing compounds which reverse the effects of 5-aminovaleric acid betaine.

In certain embodiments, this disclosure relates to methods of increasing the fat content or marbling of livestock comprising administering 5-aminovaleric acid betaine to the livestock. In certain embodiments, this disclosure relates to livestock feed compositions comprising exogenously added 5-aminovaleric acid betaine.

### BRIEF DESCRIPTION OF THE SEVERAL VIEWS OF THE DRAWINGS

FIGS. 1A-C show data indicating delta-valerobetaine (VB) is a microbiome-derived mitochondrial metabolite.

FIG. 1A shows volcano-plots of ultra-high-resolution mass spectrometry of extracts from germ-free (GF, n 95=6) and conventionalized (CV, n=6) mouse liver and liver mitochondria which provide a metabolite with a mass of 160.1332 m/z in CV. High resolution mass spectroscopy is consistent with an elemental composition of VB as C₈H₁₈NO₂. The horizontal broken line is at p-FDR=0.05, and the vertical broken line is at fold-change (FC) equal two.

FIG. 1B shows targeted analysis of 160.1332 m/z the presence of this metabolite in liver, portal vein, and cecum of mice with intact microbiome but not in germ-free mice.

FIG. 1C shows the chemical structure of delta-valerobetaine (VB). VB standard was synthesized, and the structure was confirmed by ¹H-NMR spectroscopy. Co-elution and fragmentation patterns for an experimental sample and the synthesized standard were consistent. High-resolution tandem mass spectral analysis shows a characteristic fragmentation pattern associated with the trimethyl ammonium ion at 60.061 m/z.

FIGS. 2A-E show data indicating delta-valerobetaine (VB) decreases fatty acid oxidation and increases lipid accumulation in host tissues by altering carnitine shuttle metabolism and decreasing carnitine and acylcarnitine.

FIG. 2A shows data indicating VB decreases circulating carnitine in mice. Mice of each sex were given daily intraperitoneal injection with saline (n=5), VB at 10 mg/kg (n=3) or VB at 100 mg/kg (n=8) for 1 week. Kruskal-Wallis test with Dunn's multiple comparisons test to identify pairwise differences in GraphPad Prism 6.0.

FIG. 2B shows data indicating VB decreases hepatic carnitine in mice.

FIG. 2C shows data indicating mitochondrial palmitate-dependent O₂ consumption rates are inhibited by VB. HepG2 cells were incubated for 12 h without glucose, glutamine and pyruvate and studied with either vehicle or VB at 10 or 50 μM. Effect of VB is most pronounced in the spare capacity measured after addition of the uncoupler FCCP.

FIG. 2D shows data indicating VB decreases formation of labeled acetyl-CoA from labeled palmitate in cultured cells (n=3).

FIG. 2E shows data indicating VB exacerbates hepatic steatosis in male and female mice under fasted conditions as measured by triglyceride analysis. Mice of each sex were given daily intraperitoneal injection with saline (n=5) or VB at 100 mg/kg (n=5) for 3 days±fasting.

FIGS. 3A-F indicated delta-valerobetaine (VB) increases adiposity in mice and associates with obesity phenotypes in humans.

FIG. 3A shows data indicating weight of visceral adipose tissue (VAT) is increased by VB in a diet dependent manner after 8-week VB treatment. (n=5 per group, one-tailed t-test).

FIG. 3B shows data indicating weight of subcutaneous white adipose tissue (WAT) is increased by VB in a diet-dependent manner after 8-week VB treatment.

FIG. 3C shows data indicating weight of brown-adipose tissue (BAT) is increased by VB in a diet dependent manner after 8-week VB treatment in males but not females.

FIG. 3D shows data in a subclinical population of individuals without diagnosis of disease, obese individuals (n=130) have higher VB concentration than non-obese individuals (n=84) (One-tailed t-test (p=0.0213) with Welch's correction, F, DFn, Dfd: 20.79, 129, 83, p<0.0001).

FIG. 3E shows data indicating plasma VB is correlated with increased central adiposity in adults (Visceral Adipose Tissue mass, n=179, p=0.00006).

FIG. 3F shows data indicating plasma VB is positively associated with severity of hepatic steatosis in adolescents (n=74, β=0.345, p<0.02).

FIG. 4A illustrates experiments on long-term VB treatment in GF mice. Male GF mice were administered PBS or VB in conjunction with a control diet (CD) or WD for 6 weeks with body weight recorded as percentage weight gain (two-way repeat measures ANOVA with multiple comparisons per day.

FIG. 4B shows data on weight of visceral adipose tissue (VAT) in GF mice after 6 weeks.

FIG. 4C shows data on subcutaneous white adipose tissue (SubQ).

FIG. 4D shows data on brown adipose tissue (BAT).

FIG. 4E shows data on hepatic steatosis based on scoring from liver H&E stains from GF mice.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. An “embodiment” of this disclosure refers to an example and infers that the example is not necessarily limited to the example.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

The term “betaine” refers to a compound having the quaternary trimethyl ammonium group [(CH₃)₃N+]. The terms “5-aminovaleric acid betaine,” “delta-valerobetaine,” and “VB” all refer to the compound with the chemical name 5-(trimethylammonio)pentanoate as zwitterionic salt.

Also contemplated are the alkyl ester prodrugs such as 5-methoxy-N,N,N-trimethyl-5-oxopentan-1-aminium salts and 5-ethoxy-N,N,N-trimethyl-5-oxopentan-1-aminium salts.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in compositions over the parent drug. A prodrug may be converted into the parent drug by various mechanisms, including enzymatic processes and metabolic hydrolysis. Typical prodrugs are esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol, i.e., hydroxy group, and methoxy or ethoxy esters of a carboxylic acid group.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Subject” refers to any animal, preferably a human patient, livestock, rodent, monkey, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

### Methods and Related Compositions

In certain embodiments, this disclosure relates to methods of treating or preventing obesity comprising measuring the amount of 5-aminovaleric acid betaine in a sample from a subject optionally in combination with measuring the amount of carnitine in the sample and treating or advising the subject to take certain actions to treat or prevent obesity. In certain embodiments, treating is administering or prescribing a weight loss therapeutic or dietary formulation optionally in combination with advising a schedule of feeding, exercise and/or a sleep schedule.

In certain embodiments, this disclosure relates to methods of measuring serum 5-aminovaleric acid betaine without or with carnitine as a way to identify individuals at risk of diet/microbiome-dependent obesity and obesity-related disease

In certain embodiments, this disclosure relates to methods of measuring of 5-aminovaleric acid betaine or 5-aminovaleric acid betaine/carnitine ratio before bedtime as way to identify individuals with poor nighttime fat-burning activity.

In certain embodiments, this disclosure relates to methods measuring or determining a free fatty acid to 5-aminovaleric acid betaine ratio in the morning compared to the previous evening as way to measure 5-aminovaleric acid betaine effect on nighttime fat-burning.

In certain embodiments, this disclosure relates to methods of measuring 5-aminovaleric acid betaine in order to evaluate the effect of specific probiotic supplements and supplement formulations in a subject, thereby identifying supplements that are beneficial for treating or preventing obesity and obesity-related diseases.

In certain embodiments, this disclosure relates to methods of measuring of the abundance of bacteria producing 5-aminovaleric acid betaine in stool samples and identifying or diagnosing a subject as a risk of diet-induced obesity.

In certain embodiments, this disclosure relates to methods of using 5-aminovaleric acid betaine as a feed additive to increase fat content/marbling of livestock.

In certain embodiments, this disclosure relates to methods of using 5-aminovaleric acid betaine as a pharmacologic treatment for patients with catabolic/cachectic syndromes, such as seen in aging and cancer.

In certain embodiments, this disclosure relates to methods of diagnosing a subject for a risk level of obesity complications comprising, obtaining a sample from a subject; measuring an amount of 5-aminovaleric acid betaine in the sample; and diagnosing the subject with a risk level for of obesity complications based on the measured amount 5-aminovaleric acid betaine in the sample. In certain embodiments, the sample is blood, urine, stool sample, or matter from the small intestine. In certain embodiments, the sample is blood or urine, and the subject is diagnosed with a heightened risk of obesity complications if the measured amount of 5-aminovaleric acid betaine in the sample is greater than 0.0001, 0.001, 0.01, 0.1, or 1 mg/L.

In certain embodiments, the method further comprises recording the measured amount on a computer readable medium. In certain embodiments, the method further comprises reporting the amount to a medical professional.

In certain embodiments, measuring an amount of 5-aminovaleric acid betaine is within one, two, or three hours of instituting sleep or the average time the subject falls asleep on a daily basis. In certain embodiments, measuring an amount of 5-aminovaleric acid betaine is within one, two, or three hours of waking up.

In certain embodiments, this disclosure relates to methods of diagnosing a subject for a risk level of obesity complications comprising, obtaining a sample from a subject; measuring an amount of 5-aminovaleric acid betaine in the sample; measuring an amount of carnitine in the sample; calculating the ratio of 5-aminovaleric acid betaine to carnitine in the sample; and diagnosing the subject with a risk level for obesity complications based on the calculated ratio of 5-aminovaleric acid betaine to carnitine in the sample. In certain embodiments, the sample is blood, urine, stool sample, or matter from the small intestine.

In certain embodiments, the subject is diagnosed with a heightened risk of obesity complications if the calculated ratio of 5-aminovaleric acid betaine to carnitine is greater than 1, 2, 3, 4, 5, 10, or 20.

In certain embodiments, the method further comprises recording the measured amounts or the ratio on a computer readable medium. In certain embodiments, the method further comprises reporting the amounts or ratio to a medical professional.

In certain embodiments, measuring an amount of 5-aminovaleric acid betaine and measuring an amount of carnitine in the sample is within one, two, or three hours of instituting sleep or the average time the subject falls asleep on a daily basis.

In certain embodiments, the obesity complication is an inability to lose weight or reduce body mass index despite a calorie restricted diet, poor nighttime fat-burning activity, type 2 diabetes, heart disease, high blood pressure, stroke, gallbladder disease, fatty liver disease, high cholesterol, breast, colon, or endometrial cancer.

In certain embodiments, this disclosure relates to methods of diagnosing a subject with 5-aminovaleric acid betaine producing obesogenic microbiota or for a risk level of 5-aminovaleric acid betaine related obesity complications comprising: obtaining a sample from a subject;

measuring an amount of 5-aminovaleric acid betaine in the sample; measuring an amount of carnitine in the sample; calculating the ratio of 5-aminovaleric acid betaine to carnitine in the sample; and diagnosing the subject with a risk level for of 5-aminovaleric acid betaine related obesity complications based on the calculated ratio of 5-aminovaleric acid betaine to carnitine in the sample.

In certain embodiments, the subject is diagnosed as having a 5-aminovaleric acid betaine producing obesogenic microbiota or having a heightened risk of 5-aminovaleric acid betaine related obesity complications if the calculated ratio of 5-aminovaleric acid betaine to carnitine is greater than 1, 2, 3, 4, or 5. In certain embodiments, the sample is blood, urine, stool, or matter from the small intestine.

In certain embodiments, this disclosure relates to methods of evaluating the effects of a test compound for the ability to treat or prevent obesity complications comprising, administering a test compound to a subject; obtaining a sample from the subject; measuring an amount of 5-aminovaleric acid betaine in the sample; measuring an amount of carnitine in the sample; calculating the ratio of 5-aminovaleric acid betaine to carnitine in the sample; and evaluating whether the test compounds provides a reduced risk level for obesity complications based on a reduction of the amount of 5-aminovaleric acid betaine when compared to a normal or reference value, e.g., when compared to a subject that did not receive an administration of the test compound, or a reduction in the calculated ratio of 5-aminovaleric acid betaine to carnitine in the sample when compared to a subject that did not receive an administration of the test compound.

In certain embodiments, this disclosure relates to methods of treating or preventing cachexia, sarcopenia, wasting of skeletal muscle and adipose tissue, muscle atrophy, inflammation of adipose tissues accompanied by metabolic disarrangement, anorexia, systemic inflammation, insulin resistance, muscle protein degradation, and/or weakness, comprising administering to a subject in need thereof an effective amount of 5-aminovaleric acid betaine, prodrug, or salt thereof. In certain embodiments, this disclosure relates to diagnostic assays for monitoring 5-aminovaleric acid betaine or the ratio 5-aminovaleric acid betaine to carnitine in a sample.

In certain embodiments, this disclosure relates to methods of obtaining a sample from a subject at one point in time and measuring an amount of 5-aminovaleric acid betaine in the sample, measuring an amount of carnitine in the sample, or calculating the ratio of 5-aminovaleric acid betaine to carnitine in the sample; obtaining a sample from a subject at a second point in time and measuring an amount of 5-aminovaleric acid betaine in the sample, measuring an amount of carnitine in the sample, or calculating the ratio of 5-aminovaleric acid betaine to carnitine in the sample; and comparing the amounts or ratios at the first and second points in time thereby monitoring 5-aminovaleric acid betaine or the ratio 5-aminovaleric acid betaine to carnitine in a sample.

In certain embodiments, this disclosure relates to methods of treating or preventing cachexia comprising administering to a subject in need thereof an effective amount of 5-aminovaleric acid betaine, prodrug, or salt thereof. In certain embodiments, the subject is diagnosed with cancer and receiving chemotherapy, gastrointestinal cancer, pancreatic cancer, or any terminal cancer. In certain embodiments, the subject is diagnosed with congestive heart failure. In certain embodiments, the subject is diagnosed with chronic obstructive pulmonary disease. In certain embodiments, the subject is diagnosed with chronic kidney disease or liver disease. In certain embodiments, the subject is diagnosed with human immunodeficiency virus and/or acquired immunodeficiency syndrome.

In certain embodiments, this disclosure relates to methods of treating or preventing sarcopenia comprising administering to a subject in need thereof an effective amount of 5-aminovaleric acid betaine, prodrug, or salt thereof. In certain embodiments, wherein the subject is of an age of over 65, 70, or 75 years.

In certain embodiments, this disclosure contemplates dietary formulations with custom-blended dietary supplements resulting in pre/probiotic modulation of 5-aminovaleric acid betaine production and supplement formulations containing compounds which reverse the effects of 5-aminovaleric acid betaine.

In certain embodiments, this disclosure relates to dietary formulations comprising vitamins, minerals, amino acids, exogenously added carnitine or 5-aminovaleric acid betaine. In certain embodiments, the dietary formulation further comprises fiber, herbs, or enzymes. In certain embodiments, the dietary formulation is in the form of pills, tablet, capsules, powders, gel capsules or liquids. In certain embodiments, the dietary formulation comprises orlistat. In certain embodiments, the dietary formulation comprises bupropion and naltrexone. In certain embodiments, the dietary formulation comprises phentermine. In certain embodiments, the dietary formulation comprises phentermine and topiramate.

In certain embodiments, the dietary supplements reduce appetite, making one feel full, reduce absorption of fat, or increase fat burning. Examples include, plant extracts, garcinia cambogia extracts, caffeine, raspberry ketones, glucomannan fiber, linoleic acid, omega-6 fatty acids, forskolin, synephrine, or combinations thereof. In certain embodiments, this disclosure relates to methods of increasing the fat content or marbling of livestock comprising administering 5-aminovaleric acid betaine to the livestock. In certain embodiments, the livestock is a cow, pig, chicken, or fish. In certain embodiments, this disclosure relates to livestock feed compositions comprising exogenously added 5-aminovaleric acid betaine. In certain embodiments, livestock feed comprises corn, corn gluten, soybean meal, soybean hulls, grains, grain sorghum, bakery meal, cottonseed meal, wheat middlings, or oats.

### Microbial Metabolite Delta-Valerobetaine (VB) is a Diet-Dependent Obesogen

Obesogens are chemicals that stimulate adipogenesis and fat storage. An epidemic of obesity and obesity-related metabolic disorders is believed to be linked to the intestinal microbiome. Results of experiments reported herein indicate that the microbiome-derived metabolite, delta-valerobetaine (VB) is a diet dependent obesogen that is increased with obesity and associated with visceral adipose tissue mass in humans. VB is absent in germ-free mice and their mitochondria but present in conventionalized mice and mitochondria. Mechanistic studies show VB is produced by the microbiome and decreases mitochondrial fatty acid oxidation through decreasing cellular carnitine and mitochondrial long-chain acyl-CoAs. VB administration to mice increases visceral fat mass and exacerbates hepatic steatosis with Western diet but not control diet. Thus, VB provides a molecular target to understand and potentially manage microbiome-host symbiosis/dysbiosis in diet-dependent obesity.

Metabolomic profiling complements metagenomic methods with capability to measure metabolic products of microbiome, diet, and host interactions. This can be used for personal monitoring of intestinal microbiota functions in digestion of dietary macromolecules and synthesis of diverse metabolites that directly impact human metabolism. Epidemiological and experimental evidence indicates microbial products such as lipopolysaccharide, phenylacetic acid, and methylamines contribute to development of insulin resistance, hepatic steatosis, and cardiovascular disease. Untargeted metabolomic analyses of germ-free (GF) and control (C) mice with a reconstituted or intact microbiome further reveal approximately 10% of circulating mammalian metabolome is of microbial origin; the majority of these metabolites are unidentified and without known functions.

Among the known functions of microbiome-derived metabolites, quorum sensing compounds support communication within and between bacterial species. Recent evidence for bi-directional communication between the microbiome and host mitochondria in metabolic health and disease raises the possibility that specific chemical signals also mediate microbiome-host mitochondria symbiosis.

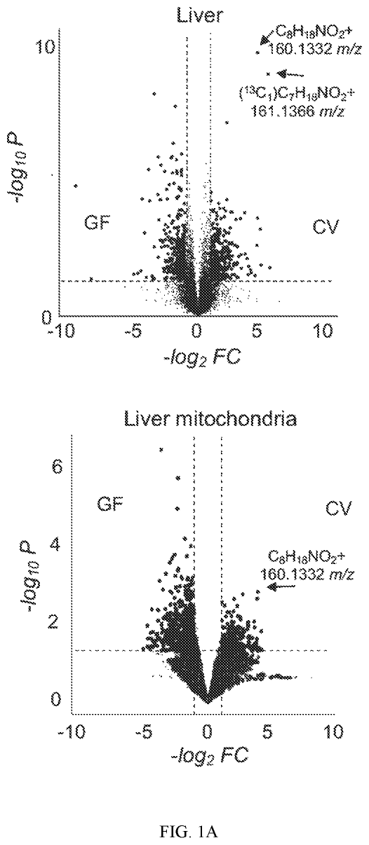

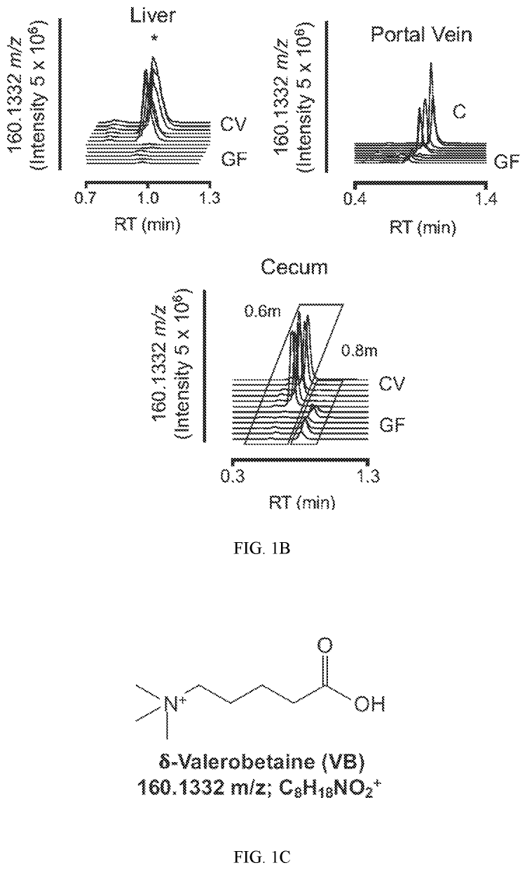

To identify microbial metabolites that could impact mitochondrial function, the metabolome of liver and liver mitochondria was examined in mice with an intact microbiome (“conventionalized” mice in which germ-free mice were reconstituted with microbiome) compared to liver and liver mitochondria from germ-free mice (FIG. 1A). The top discriminatory metabolite has an accurate mass m/z of 160.1332 by ultra-high-resolution mass spectrometry, corresponding to predicted elemental composition C₈H₁₈NO₂. This metabolite is found in liver and other tissues, portal and peripheral circulation, and cecum of conventional but not germ-free mice (FIG. 1B). Authentic standard co-elution and MS/MS experiments show the identity of 160.1332 m/z to be delta-valerobetaine (VB) (FIG. 1C). Concurrent analyses of propionylcholine and valine betaine, similar molecules with identical elemental compositions, conclusively excluded these structures. VB was not present in the mouse diet and was not detectable in other samples collected from GF mice. Concentrations of VB in conventional mice were in the millimolar range in the cecum, with somewhat lower concentrations in the colon. Concentrations were 9-26 μM in the portal vein, with higher concentrations in the liver (75-190 μmol/kg wet weight) and lower concentrations in the peripheral plasma (2-10 μM) indicating tissue accumulation.

To determine whether intestinal microbes produce VB, ex vivo incubations of cecum contents from conventional and GF mice were performed. LC-MS/MS analyses indicated that only conventional cecum contents produce VB whereas GF cecum contents do not. Incubations of candidate microbes from diverse taxa of gram positive and gram negative pathogenic and commensal bacteria, including LGG, E. coli, Salmonella typhimurium (SL1344) and other lactobacilli and lactococci, show VB is a common metabolite of intestinal microbes. The presence of VB in diverse tissues and mitochondria of mice with intact microbiome therefore reflects microbial production, followed by absorption and distribution.

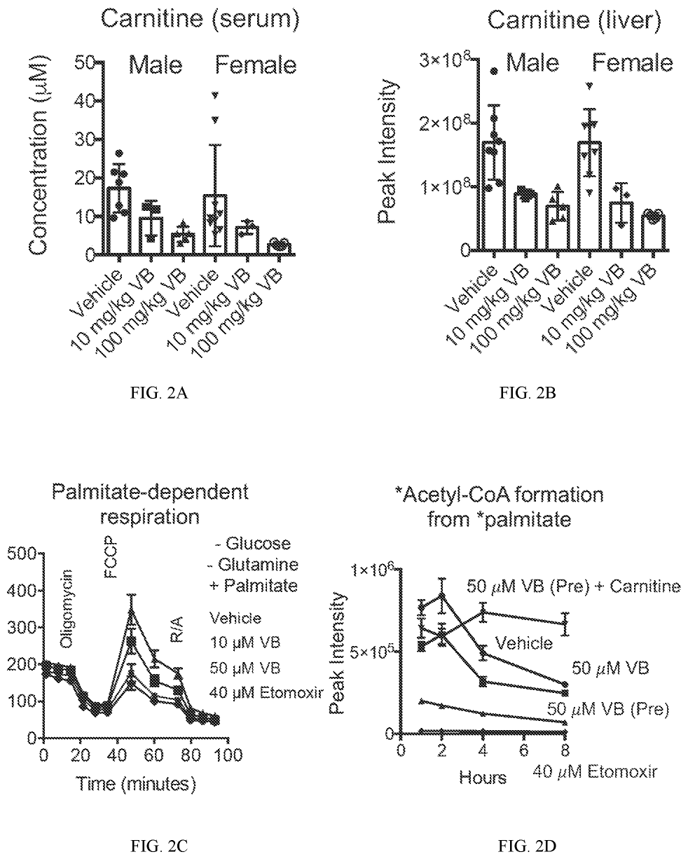

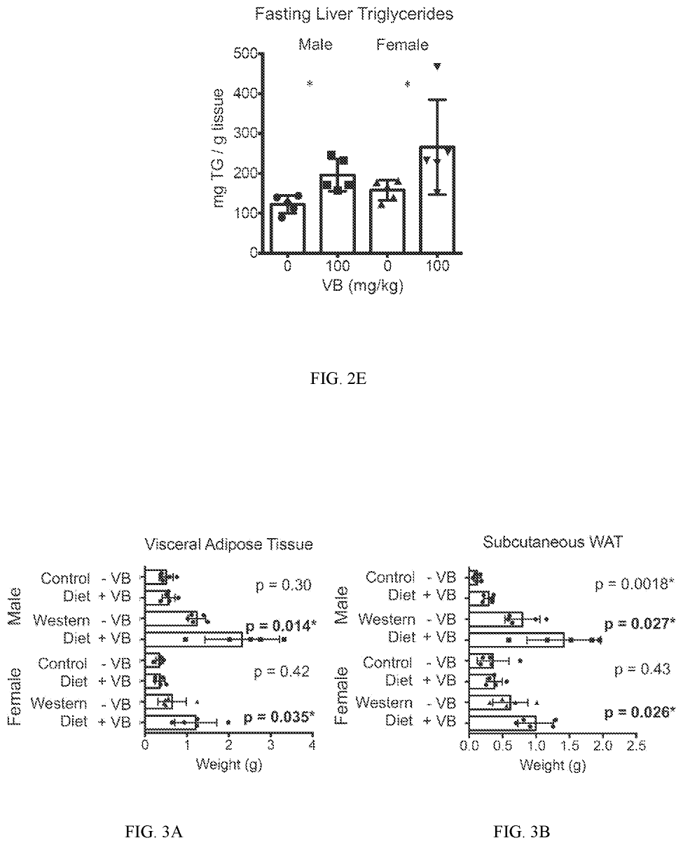

Dose-response experiments with VB were performed in vitro and in vivo to examine metabolic effects of VB. The mitochondrial carnitine shuttle is the top metabolic pathway associated with VB in human HepG2 cells. At 10 μM, equivalent to VB present in conventional mouse portal circulation, the cell content of carnitine is decreased to half that of control cells. The carnitine shuttle uses carnitine for transport of long-chain fatty acyl chains into mitochondria for fatty acid β-oxidation. Under culture conditions mimicking fasting, VB elicits a dose-dependent decrease in palmitate-dependent mitochondrial oxygen respiration (FIG. 2C). The effect of VB on inhibition of palmitate-dependent oxygen consumption rate is confirmed by stable isotope tracer studies of palmitate metabolism, which show VB inhibits formation of ¹³C₁₆ palmitoylcarnitine from ¹³C₁₆ palmitate and decreases the formation of ¹³C₂ acetyl-CoA (FIG. 2D). Rescue experiments show addition of carnitine restores cellular carnitine and formation of palmitoylcarnitine. Thus, VB inhibits mitochondrial long chain fatty acid metabolism through effects on carnitine and operation of the carnitine shuttle. Control experiments for studies of palmitate-dependent O₂ consumption rate show that when glucose, glutamine, and pyruvate are present at high concentration in the culture media, VB causes little decrease in basal O₂ consumption rate, ADP-linked O₂ consumption rate, spare capacity, or non-mitochondrial respiration. This shows the effect of VB on fatty acid β-oxidation for mitochondrial energy production is dependent on the availability of other fuel substrates.

As observed in cells, the carnitine shuttle is the top metabolic pathway associated with VB-treatment in mice. VB treatment decreases circulating (FIG. 2A), hepatic (FIG. 2B), heart, and brain carnitine. Furthermore, VB treatment alters circulating and systemic acylcarnitine and acyl-CoA profiles. In whole tissue, VB increases hepatic and cardiac palmitoyl-CoA and decreases other short, medium, and long-chain acylcarnitines and CoAs under fasted conditions. In mitochondria, VB decreases the abundance of long-chain fatty acyl-CoAs and carnitines, consistent with a decrease in long-chain fatty acid β oxidation. VB also decreases circulating and hepatic β-hydroxybutyrate, a product of mitochondrial β-fatty acid oxidation. Liver triglycerides increase after fasting in both male and female mice (FIG. 2E), and Oil Red O staining of liver shows VB-treatment increases lipid deposits, especially under fasted conditions. Lipid profiling shows VB treatment increases triacylglycerol and diacylglycerol species in liver, heart, and brain. Taken together, these data show VB causes systemic carnitine insufficiency, resulting in a decrease in mitochondrial long-chain fatty acid β-oxidation and tissue fat accumulation.

To further understand VB orchestration of microbiome mitochondria interactions regulating host metabolism, transcriptome and metabolome profiles from GF and CV mouse livers were analyzed. Because VB, which is absent in GF mice, causes an accumulation of long chain acyl-CoAs and triglycerides in liver, it was anticipated that hepatic transcriptome profiles of CV mice would reflect alterations to mitochondrial function, with increases in abundance of transcripts for TAG synthesis, lipoprotein synthesis and export. Overrepresentation analysis (ORA) of the transcriptome data reveals mitochondria (GO:CC, p-FDR<10-150) and lipid metabolism/transport (Reactome, p-FDR<10-50) as top host pathways associated with intact microbiome. More specifically, genes encoding mitochondrial energy production and fatty acid oxidation [Sdh, Nduf, Atp5f, Cycl, Cox5a, Vdac, Hadh, Acsl, Eci, Acaa], TAG biosynthesis [Agpat, Dgat2, Gpat3], and lipoprotein export [Apoa, Apob, Apoc, Apoe, Mttp, Pdia] are increased in conventionalized mice. Furthermore, integration of transcriptome and metabolome pathways related to energy metabolism shows the microbiome elicits a metabolic shift towards mitochondria and lipid pathways. In contrast, germ free mice have amino acid and carbohydrate metabolism as top pathways. Taken together, these results indicate microbial production of VB inhibits hepatic mitochondrial β-oxidation, increases hepatic TAG synthesis, and causes fatty liver, especially in a fasted state. VB effects in fasting may be particularly informative as a mechanism causing obesity because decreasing mitochondrial β-oxidation of adipose-derived lipids during fasting could cause long-term increase in adiposity.

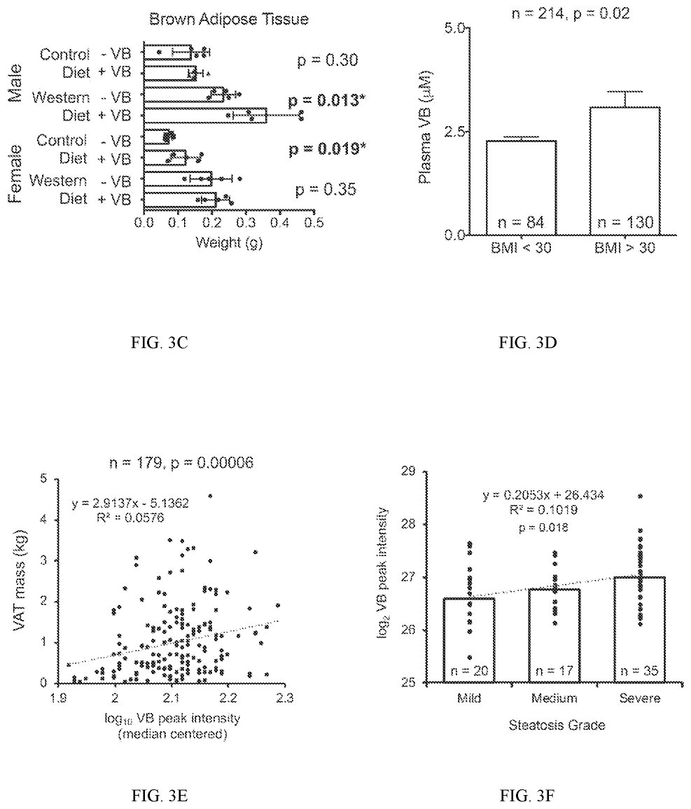

Experiments indicate VB increases adipose tissue fat mass in an 8-week study of mice, with substantial increases in visceral adipose tissue (VAT) (FIG. 3A) and subcutaneous white adipose tissue (WAT) (FIG. 3B) in mice fed a Western diet. The relative increases are most pronounced in VAT, where fat mass in VB-treated mice on a Western diet was increased 80% in male mice and 60% in female mice compared to Western diet alone. VB also increases brown adipose tissue (BAT) but in a less consistent manner (FIG. 3C). Trends for increase in total body mass occur in VB-treated mice with the Western Diet, but these did not have p<0.05 after 8 weeks. The long-term consequences of VB inhibition of mitochondrial β-oxidation of fatty acids therefore include effects on body composition in mice receiving Western diet, with specific and pronounced effects on visceral adipose tissue mass.

To test for VB association with obesity in humans, plasma VB was measured in (34 male, 96 female, mean age 53 y) obese individuals [Body Mass Index (BMI)>30 kg/m²] without overt disease compared to 84 (20 male, 64 female, mean age 53 y) individuals from the same cohort with BMI<30. The results show average VB concentration in the obese individuals is 40% higher than non-obese individuals (FIG. 3D). Plasma VB in 179 subjects (63 males, 116 females, mean age 50 y) shows a positive correlation with visceral adipose tissue (VAT) mass (β=3.7E+04±1.1E+04), independent of age, race, and sex (FIG. 3E). This relationship is also maintained when controlling for total body fat, establishing a relationship between VB and both visceral adipose tissue mass and BMI in humans. Similarly, VB was measured in plasma from a cross-sectional study of youth with biopsy-proven NAFLD (n=74, mean age 14 y, males=54, females=20) and an association of plasma VB was found with hepatic steatosis (FIG. 3F). Circulating VB levels were 40% higher with severe steatosis (>66% steatosis) compared to mild steatosis (<33% steatosis; β=0.328, p=0.03, linear regression adjusted for age, sex, and race).

The possible dependence of these relationships upon diet, as found in mice, cannot be directly tested in these retrospective human studies. Evidence for common mechanisms is provided, however, by correlations between plasma VB and plasma carnitine in the human populations. Non-obese individuals have positive correlation of plasma VB and carnitine (Spearman r=0.33 and 0.45 for studies in FIG. 3D, 3E) while obese individuals have weakly negative or no correlation (r=−0.08). MWAS shows VB associates with carnitine shuttle metabolism in non-obese subjects. In obese subjects, VB associates with arachidonate, leukotriene, methionine/cysteine, and purine metabolism. The latter associations occur with inflammation and oxidative stress, both of which contribute to insulin resistance, a frequent characteristic of visceral adiposity. Homeostasis model assessment of insulin resistance (HOMA-IR) for the individuals with VAT measures studied in FIG. 3E show HOMA-IR is positively associated with VB. These results indicate additional pathogenic mechanisms accompany VB-associated obesity phenotypes in humans.

Systemic accumulation of lipids occurring as a result of VB modulation of carnitine dependent fatty acid oxidation may reflect an evolutionarily conserved benefit of microbiome mitochondria communication in energy metabolism and conservation. Phylogenetic and evolutionary evidence show eukaryotic mitochondria are descendent from an ancient bacterial endosymbiont. There is increasing recognition that the eukaryotic host and its associated microbiome function as a biological unit, termed holobiont, and are subject to evolutionary pressures. In calorie-restricted states, microbial production of VB could promote survival of the holobiont/symbiont by functioning as a brake on host fatty acid oxidation, preserving collective nutritional resources. In support of this, GF-mice, which are protected from diet-induced obesity, succumb more quickly following prolonged fasting periods compared to conventional mice. In the context of overnutrition provided by the modern Western diet, an evolved symbiotic preservation of nutritional resources provided by VB could drive diet-dependent obesity. In recognition of this, it cannot pass without notice that improved quality of maize with high lysine content, which is helping to erase global malnutrition, may have contributed to the global obesity epidemic through increased content of the VB precursor, trimethyllysine, and shift in this evolved holobiont/symbiont relationship.

An alternative interpretation of VB activity involves an adaptive response termed mitohormesis, a process by which a minor impairment increases gene expression of mitochondrial systems which improve metabolic function of mitochondria. Our analysis of GF/CV mouse liver transcriptomes shows the microbiome increases expression of many genes for mitochondrial bioenergetic and lipid homeostasis. With this interpretation, VB-induced lipid accumulation could drive activity of SIRT1, PGC-1a and PPAR-a pathways which control carnitine homeostasis, mitochondrial bioenergetics, and lipid metabolism. PPAR-a expression declines with age, and VB elicits a dose-dependent increase in PPAR-response element-linked luciferase activity. Thus, VB may function through a mitohormesis response mechanism and in conjunction with diet and aging, contribute to age dependent changes in adiposity.

Use of clinical VB measures to guide diet and health management includes consideration of these complex holobiont/symbiont and mitohormesis mechanisms and likely concurrent microbiome and mitochondrial function tests. VB from dietary sources has both positive and negative health implications. VB from ruminant microbes is relatively high in ruminants and improves nutritional value of milk by increasing acylcarnitine content. Conversely, trimethyllysine, a precursor to VB, is high in foods (e.g., red meat) associated with poor metabolic health and increased in people at risk for cardiovascular diseases. Whole grain diets, which do not contain VB but increase circulating VB in humans through pre-biotic effects on gut microbial composition such as increasing Lactobacillus and increasing the Bacteroidetes/Firmicutes ratio, are associated with decreased risk of cardiometabolic disorders, type 2 diabetes, and weight gain. Therefore, effective use of VB measures in management of obesity will require evaluation in the context of diet and microbiome. This interpretation is consistent with potential use for NAFLD, where circulating VB is increased and hepatosteatosis is potentiated by a high-fat diet.

Interactions of VB with the carnitine shuttle has widespread implications for human health. Carnitine regulates glucose and lipid metabolism, and free carnitine declines are accompanied by an accumulation of medium and long-chain acylcarnitines in muscle and obesity in aging. Carnitine supplementation is beneficial for obesity, fatty liver, and glucose utilization. Accumulation of incompletely oxidized acylcarnitines is associated with obesity and insulin resistance, potentially exacerbated by chemicals that decrease cellular carnitine, cause mitochondrial dysfunction, and decrease hepatic fatty acid oxidation. Therefore, assessment of interactions of VB with the carnitine system could simplify use of VB measures within the context of holobiont/symbiont and mitohormesis mechanisms.

Delta-valerobetaine, a structural analogue of the carnitine precursor γ-butyrobetaine, is a gut microbe-derived molecule which orchestrates microbiome-mitochondrial communication to control energy metabolism and cause visceral adiposity. Measures of VB, along with carnitine, provide an approach to guide use of prebiotic and probiotic interventions and dietary management for excessive adiposity and associated pathogenic mechanisms.

### VB Treatment Worsens Western Diet (WD)-Induced Adiposity in Mice

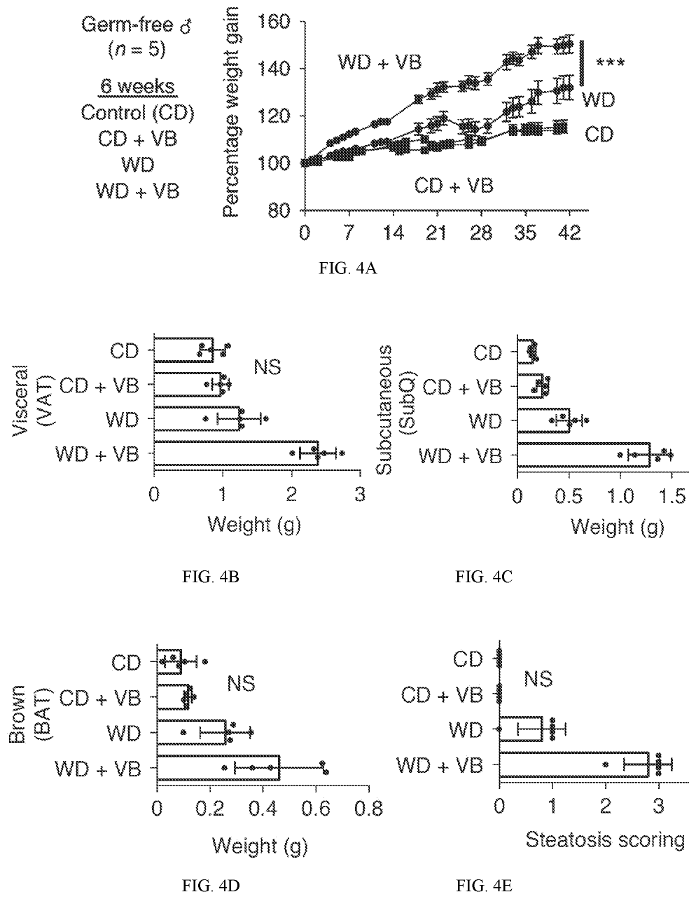

VB effects in fasting are particularly intriguing as a mechanism causing obesity because decreasing mitochondrial β-oxidation of lipids mobilized from adipose tissue during fasting could cause a long-term increase in fat deposition. Therefore, experiments were performed to determine whether long-term VB treatment could increase weight gain and adiposity in mice. This was tested this in both GF and conventional mice, with or without a high-fat/sugar WD. Backhead et al. report a resistance to diet-induced obesity in germ-free mice. PNSA, 104(3), 2007. In GF mice, VB was dosed parenterally to achieve concentrations similar to VB concentrations observed in untreated conventional mice, which also resulted in carnitine concentrations equivalent to conventional mice. Six weeks of VB treatment increased weight gain in GF mice fed a WD (FIG. 4A), characterized by increased adipose tissue fat mass, with substantial increases in perigonadal visceral (VAT), posterior subcutaneous (SubQ) and interscapular brown-adipose (BAT) compartments compared to GF mice administered a WD alone. Furthermore, VB treatment exacerbated the development of hepatic steatosis in these mice (FIG. 4E). Similarly, VB increased adipose tissue fat mass (VAT, SubQ, BAT) in conventional mice after 8 weeks with the WD. The relative increases were most pronounced in white adipose compartments (VAT, SubQ) where fat mass in VB treated GF and conventional mice on a WD was increased over 80% compared to WD alone.

Trends for increase in severity of hepatic steatosis and total body mass were observed in VB treated conventional mice with the WD, and while there was a significant increase in body mass observed in the first 10 days of the experiment, these did not remain significantly different after 8 weeks. Although WD-fed mice gained more weight than control fed mice, no differences in circulating VB were observed between WD or control-diet-fed conventional mice. These effects were not observed in GF or conventional mice fed the control diet.

1. A method of diagnosing a subject for a risk level of obesity complications comprising:
obtaining a sample from a subject;
measuring an amount of 5-aminovaleric acid betaine in the sample; and
diagnosing the subject with a risk level for of obesity complications based on the measured amount 5-aminovaleric acid betaine in the sample.

2. The method of claim 1, wherein the sample is blood, urine, stool sample, or matter from the small intestine.

3. The method of claim 1, wherein the sample is blood or urine and the subject is diagnosed with a heightened risk of obesity complications if the measured amount of 5-aminovaleric acid betaine in the sample is greater than 0.0001 mg/L.

4. The method of claim 1, wherein measuring an amount of 5-aminovaleric acid betaine is within one hour of instituting sleep.

5. The method of claim 1, further comprising
measuring an amount of carnitine in the sample;
calculating the ratio of 5-aminovaleric acid betaine to carnitine in the sample; and
diagnosing the subject with a risk level for obesity complications based on the calculated ratio of 5-aminovaleric acid betaine to carnitine in the sample.

6. The method of claim 5, wherein the sample is blood, urine, stool sample, or matter from the small intestine.

7. The method of claim 5, wherein the subject is diagnosed with a heightened risk of obesity complications if the calculated ratio of 5-aminovaleric acid betaine to carnitine is greater than 1.

8. The method of claim 5, wherein measuring an amount of 5-aminovaleric acid betaine and measuring an amount of carnitine in the sample is within one hour of instituting sleep.

9. A method of treating or preventing cachexia comprising administering to a subject in need thereof an effective amount of 5-aminovaleric acid betaine, prodrug, or salt thereof.

10. The method of claim 9, wherein the subject is diagnosed with gastrointestinal cancer, pancreatic cancer, or any terminal cancer.

11. The method of claim 9, wherein the subject is diagnosed with congestive heart failure.

12. The method of claim 9, wherein the subject is diagnosed with chronic obstructive pulmonary disease.

13. The method of claim 9, wherein the subject is diagnosed with chronic kidney disease.

14. The method of claim 9, wherein the subject is diagnosed with human immunodeficiency virus and/or acquired immunodeficiency syndrome.

15. A method of increasing the fat content or marbling of livestock comprising administering 5-aminovaleric acid betaine to the livestock.

16. The method of claim 15, wherein the livestock is cow, pig, chicken, or fish.

17. A livestock feed comprising exogenously added 5-aminovaleric acid betaine for use in the method of claim 15.
